(19) United States
(12) Patent Application Publication
(10) Pub. No.: US 2015/0231090 A1
(43) Pub. Date: Aug. 20, 2015

(54) USAGE OF GUTTIFERONE K FOR TREATING HIGH METASTATIC CANCER

(71) Applicants: Hong Kong Baptist University, Hong Kong (HK); Shanghai University of Traditional Chinese Medicine, Shanghai (CN)

(72) Inventors: Hongxi XU, Hong Kong (HK); Kaikai SHEN, Shanghai (CN); Yuanzhi LAO, Shanghai (CN); Hongsheng TAN, Shanghai (CN); Kaixian CHEN, Shanghai (CN); Dajian YANG, Chongqing (CN); Shilin CHEN, Beijing (CN); Zhaoxiang BIAN, Hong Kong (HK); Chengyuan LIN, Hong Kong (HK); Aiping LU, Hong Kong (HK); Albert Sun Chi CHAN, Hong Kong (HK)

(21) Appl. No.: 14/701,529

(22) Filed: May 1, 2015

Related U.S. Application Data
(63) Continuation-in-part of application No. 14/149,821, filed on Jan. 8, 2014.
(60) Provisional application No. 61/750,969, filed on Jan. 10, 2013.

(51) Int. Cl. A61K 31/122; A61K 36/38

(52) U.S. Cl. CPC A61K 31/122; A61K 36/38

(57) ABSTRACT

The present invention provides composition for treating cancer comprising polyprenylated acylphloroglucinol (PPAP) compound. The present invention also provides a composition comprising Guttiferone K for treating esophageal cancer and liver cancer. The present invention further provides a method of using said compound for inhibiting cancer metastasis.

### CROSS-REFERENCE TO RELATED APPLICATIONS

The present application is a continuation-in-part application of the U.S. non-provisional patent application Ser. No. 14/149,821 filed Jan. 8, 2014, which claims priority of U.S. Provisional Patent Application Ser. No. 61/750,969 filed Jan. 10, 2013, and which the disclosures are hereby incorporated by reference in its entirety.

### FIELD OF INVENTION

This invention relates to the use of a chemical entity for therapeutic uses. More particularly, it relates to the use of a compound with anticancer effects on metastatic cancer.

### BACKGROUND OF INVENTION

Esophageal cancer is one of the most common malignancies and is associated with a dismal prognosis. Although treatment options have increased for some patients, overall progress has been modest. Another common malignancy is liver cancer. The most frequent liver cancer, accounting for approximately 75% of all primary liver cancers, is hepatocellular carcinoma (HCC) (also named hepatoma). HCC is a cancer formed by liver cells, known as hepatocytes that become malignant. Another type of cancer formed by liver cells is hepatoblastoma, which is specifically formed by immature liver cells. It is a rare malignant tumor that primarily develops in children, and accounts for approximately 1% of all cancers in children and 79% of all primary liver cancers under the age of 15. Most hepatoblastomas form in the right lobe.

Liver cancer can also form from other structures within the liver such as the bile duct, blood vessels and immune cells. Cancer of the bile duct (cholangiocarcinoma and cholangiocellular cystadenocarcinoma) account for approximately 6% of primary liver cancers. There is also a variant type of HCC that consists of both HCC and cholangiocarcinoma. Tumors of the blood vessels (angiosarcoma and hemangioendothelioma, embryonal sarcoma and fibrosarcoma are produced from a type of connective tissue known as mesenchyme. Cancers produced from muscle in the liver are leiomyosarcoma and rhabdomyosarcoma. Other less common liver cancers include carcinosarcomas, teratomas, yolk sac tumours, carcinoid tumours and lymphomas. Lymphomas usually have diffuse infiltration to liver, but it may also form a liver mass in rare occasions.

Many cancers found within the liver are not true liver cancers, but are cancers from other sites in the body that have spread to the liver (known as metastases). Frequently, the site of origin is the gastrointestinal tract (such as colon cancer and carcinoid tumors mainly of the appendix), but also from breast cancer, ovarian cancer, lung cancer, renal cancer, prostate cancer.

Thus, there is a great need to develop new treatments. The present invention provides a compound, Guttiferone K, and method of using the same for treating liver cancer by inhibiting the migration and invasion of HCC cancer.

In the last decade, most of the research on Garcinia species has focused on the anticancer activity of gambogic acid (GA), a caged xanthone found at high concentrations in gamboge. GA has been involved in the injectable anticancer drug since the 1970s. In 2004, GA has been granted permission for testing in clinical trial as a wide spectrum anticancer drug. Gambogic acid and its derivatives are cytotoxic in many cancer cell lines by binding to the transferrin receptor and induction of G₂/M cell cycle arrest and mitochondrial and death receptor-mediated apoptosis. Gambogic acid also reduces invasion and angiogenesis, telomerase mRNA expression and activity and tumor volume in vivo. However, the anticancer effect of gambogic acid is not selective and it induces toxicity to the liver and kidney, which limits its development into a clinically useful anticancer drug.

Due to the toxicity of gambogic acid, there is a need for a novel and more selective compounds isolated from various Garcinia species for cancer treatment. This is an objective of this invention.

Citation or identification of any reference in this section or any other section of this application shall not be construed as an admission that such reference is available as prior art for the present application.

### SUMMARY OF INVENTION

Accordingly, the objective of this invention is to provide a compound that exhibits potent anticancer effect and method of using the same for use in cancer treatment.

In accordance with a first aspect of the present invention, there is provided an anti-cancer composition comprising an effective amount of compound of Formula I,

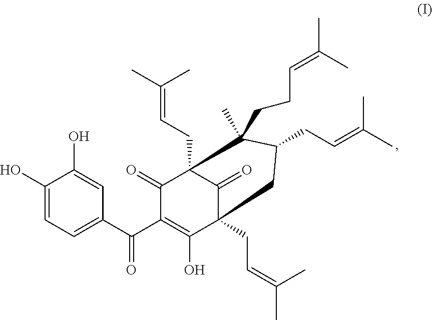 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]

for treating metastatic cancer comprising esophageal cancer and having anti-cancer activities comprising inhibiting migration and invasion of the esophageal cancer.

In accordance with a second aspect of the present invention, there is provided a method for treating cancer comprising administering to a subject in need of a composition comprising an effective amount of a compound with a chemical structure of Formula I:

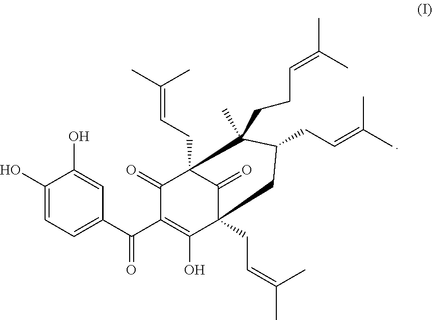 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]

In a first embodiment of the first aspect of the present invention, said composition comprising Guttiferone K.

In a second embodiment of the first aspect of the present invention, the cancer comprises esophageal cancers and liver cancers.

In a third embodiment of the first aspect of the present invention, the compound has anti-cancer activities comprising anti-migration of cancer cells and anti-invasion of cancer cells.

In a fourth embodiment of the first aspect of the present invention, the effective amount of compound of Formula I administered is more than 1 mg/10 kg of subject's body weight.

In a fifth embodiment of the first aspect of the present invention, the effective amount of the compound of Formula I administered to said subject ranges from 3 mg per 10 kg to 10 mg per 10 kg of the subject's body weight.

In a sixth embodiment of the first aspect of the present invention, the composition is administered via injection, including intraperitoneal, intravenous and subcutaneous injection.

In a seventh embodiment of the first aspect of the present invention, the cancer being treated is metastatic.

In a second aspect of the present invention there is provided a method of using the compound of Formula I for cancer treatment.

In a first embodiment of the second aspect of the present invention, said compound of Formula I is used as a cancer metastasis inhibitor.

In a second embodiment of the second aspect of the present invention, the cancer being treated comprises esophageal cancers and liver cancers.

In a third embodiment of the first aspect of the present invention, the cancer being treated is metastatic.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of Guttiferone K.

FIG. 2 shows the wound healing assay of Guttiferone K in ECA109 cells.

FIG. 3 shows the transwell assay of Guttiferone K in ECA109 cells. FIG. 3A is the statistical assay of migration cells in different Guttiferone K concentration treatment. FIG. 3B is the giemsa staining of migration cells in different Guttiferone K concentration treatment.

FIG. 4 shows the matrigel assay of Guttiferone K in ECA109 cells. FIG. 4A is the statistical assay of invasion cells in different Guttiferone K concentration treatment. FIG. 4B is the giemsa staining of invasion cells in different Guttiferone K concentration treatment.

FIG. 5 shows the effect of Guttiferone K on HCC metastasis in vitro and in vivo. Cell migration activity (shown in FIGS. 5A (microscope images) and 5B (bar chart)) and cell invasion activity (shown in FIGS. 5C (microscope images) and 5D (bar chart)) after treatment with Guttiferone K (0-20 μM) in HepG2 cells for 24 h and 48 h. Data are shown as means±SEM; *p<0.05, ***p<0.001 compared with control. n=3 (D-G) After injected with HepG2 cells (1×10⁶ cells per mouse) via tail vein, BALB/c nude mice are i.p. injected with Guttiferone K (1 mg/kg, 3 mg/kg, and 10 mg/kg) every other day. On day 28, the mice are killed. FIG. 5E shows schedule of mouse studies. FIG. 5F shows upper panel lungs were fixed in Bouin's buffer and photographed. Lower panel lungs are fixed in 4% paraformaldehyde (PFA), and sectioned for H&E staining. Arrowheads indicate tumor metastasis nodules. a: Control; b: Vehicle; c: Guttiferone K 1 mg/kg; d: Guttiferone K 3 mg/kg; e: Guttiferone K 10 mg/kg. Scale bar=1 mm. FIG. 5G shows statistics of lung metastasis. FIG. 5H shows the body weight recorded every third day. Data are shown as means±SEM; ^{###}p<0.001 Vehicle compared with Control; *p<0.05, **p<0.01, ***p<0.001 compared with Vehicle. n=8

FIG. 6 shows the toxicity of Guttiferone K in vivo. BALB/c male mice are randomly divided into 5 groups: Control, Vehicle Control, Guttiferone K treatment (1, 3, 10 mg/kg) for 28 days by intraperitoneal injection (n=8 mice in each group). FIG. 6A and FIG. 6B show the HE staining sections (in FIG. 6A, scale bar=1 mm) or apparent changes in appearance (in FIG. 6B, scale bar=1 cm) in multiple organs in Guttiferone K treatment group. FIG. 6C shows no evidence of weight loss in treatment of Guttiferone K group.

FIG. 7 shows the cytotoxicity of Guttiferone K in vitro. HepG2 cells are seeded in 6 wells plate and live cell counting was applied to test the cytotoxicity. FIG. 7A and FIG. 7B show the treatment of Guttiferone K at indicated dose for 24 and 48 hours respectively and does not cause significant cell death in HepG2 cells.

### DETAILED DESCRIPTION OF INVENTION

The present invention is not to be limited in scope by any of the specific embodiments described herein. The following embodiments are presented for exemplification only.

Garcinia species (Family Guttiferae) are tropical evergreen trees and shrubs that are widely distributed in Southeastern Asia and their phytochemistry has been widely studied. Classic and caged xanthones have been isolated from various parts of these plants, and identified as their major bioactive components. Traditionally, Garcinia extract (called gamboge) has been used in folk and Chinese medicine to promote detoxification and treat inflammation and wounds, and xanthones isolated from various Garcinia species also showed antibacterial, antioxidant, antiviral and neuroprotective effects in Reutrakul, V.; Anantachoke, N.; Pohmakotr, M.; Jaipetch, T.; Sophasan, S.; Yoosook, C.; Kasisit, J.; Napaswat, C.; Santisuk, T.; Tuchinda, P., Cytotoxic and anti-HIV-1 caged xanthones from the resin and fruits of Garcinia hanburyi. Planta Med 2007, 73 (1), 33-40; and (a) Rukachaisirikul, V.; Phainuphong, P.; Sukpondma, Y.; Phongpaichit, S.; Taylor, W. C., Antibacterial caged-tetraprenylated xanthones from the stem bark of Garcinia scortechinii. Planta Med 2005, 71 (2), 165-70; (b) Jang, S. W.; Okada, M.; Sayeed, I.; Xiao, G.; Stein, D.; Jin, P.; Ye, K., Gambogic amide, a selective agonist for TrkA receptor that possesses robust neurotrophic activity, prevents neuronal cell death. Proc Natl Acad Sci USA 2007, 104 (41), 16329-34; (c) Sampath, P. D.; Kannan, V., Mitigation of mitochondrial dysfunction and regulation of eNOS expression during experimental myocardial necrosis by alpha-mangostin, a xanthonic derivative from Garcinia mangostana. Drug Chem Toxicol 2009, 32 (4), 344-52.

It is found by the inventors of the subject application that several polyprenylated acylphloroglucinol (PPAP) compounds had potent cytotoxic effects on human colorectal cancer cell lines without affecting the normal human colon fibroblasts. On human colon cancer HT-29 cell line, Guttiferone K is the most potent PPAP compound. The present invention provides a compound, Guttiferone K for use as an anticancer drug against esophageal cancer by significantly inhibiting esophageal cancer cell migration and invasion.

Extraction and Isolation.

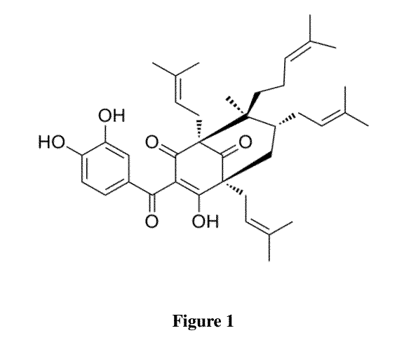

The air-dried and powdered pericarp (9.0 kg) of Garcinia yunnanensis Hu is extracted with acetone (20 L) at room temperature for three times. The extracted solution was evaporated under reduced pressure to yield a dark green residue (1.2 kg). The residue was chromatographed on silica gel eluted by CHCl₃, EtOAc, and acetone sequentially. The CHCl₃ fraction is evaporated in vacuum to give a residue (750 gram), part of which (400 gram) is subjected to silica gel column eluted with a gradient hexane/acetone system (100:0 to 0:100, v/v). Four fractions (I-V) are obtained on the basis of TLC analysis. Fraction II is separated using silica gel and eluted with gradient petroleum ether/acetone system (10:0 to 0:10) to produce four subfractions. The second subfraction is separated by preparative HPLC on an Alltima C-18 column eluted with CH₃CN in 0.1 acetic acid (0.1% acetic acid/CH3CN, 5/95) to yield Guttiferone K (3.0 g). The structure of Guttiferone K (Formula I) is shown in FIG. 1.

Cell Culture

Human esophageal cancer cell line ECA109 are maintained in RPMI1640 (Invitrogen) supplemented with 10% fetal bovine serum (Invitrogen), 100 U/ml penicillin and 100 μg/ml streptomycin, within a humidified atmosphere containing 5% CO2 at 37° C.

Anti-Migration Activity

Wound Healing Assay

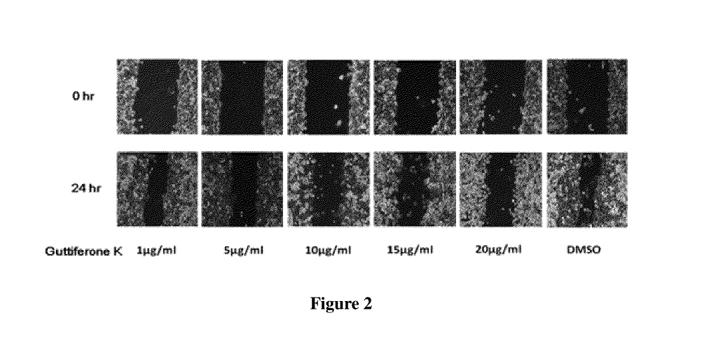

ECA109 cells are seeded on 12-well plates at a density of 1×10⁵ cells/well. After the cells reached sub-confluence, the mono-layer cells are wounded by scraping off the cells and then grown in medium for 24 hours. The migrated distance of cells is monitored and imaged under a microscope. As shown in FIG. 2, Guttiferone K suppresses the wound healing in a concentration dependent manner.

Transwell Assay

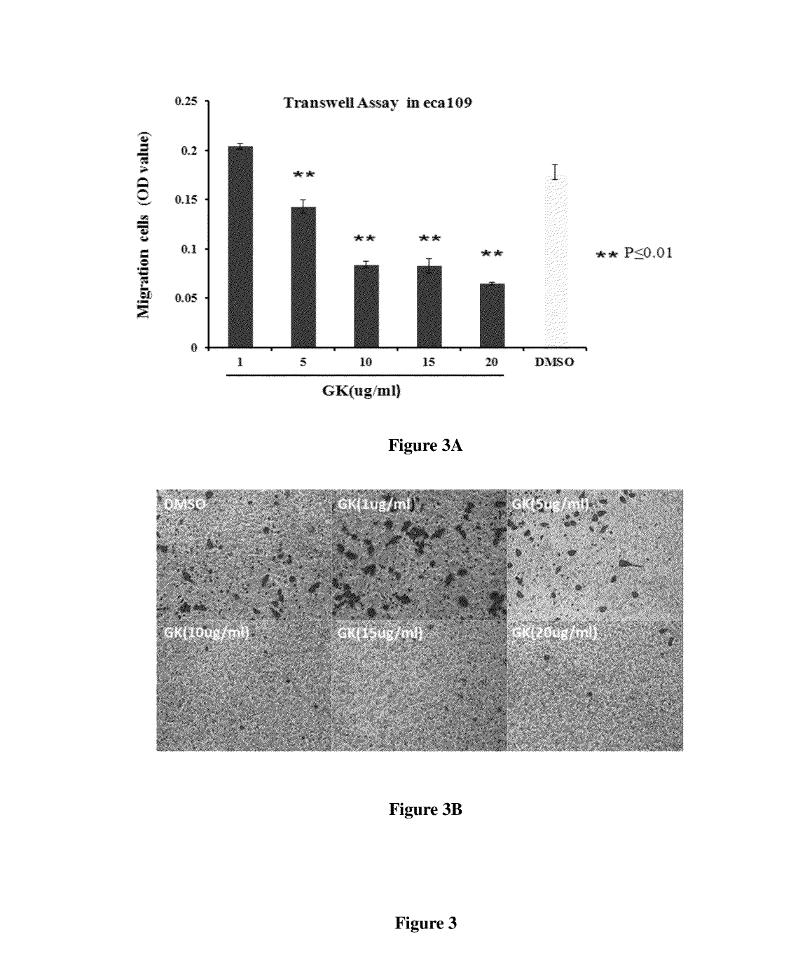

Cell migration is also determined using a transwell (Corning) with a pore size of 8 μm. 5×10⁴ cells are seeded in serum-free medium in the upper chamber, while medium containing 10% FBS in the lower chamber. After incubating for 24 hours at 37° C., cells in the upper chamber are carefully removed with a cotton swab and the cells that have traversed to reverse face of the membrane are fixed in methanol, stained with Giemsa, and counted. FIG. 3 shows the Guttiferone K suppresses ECA109 cells migration through transwell at concentration dependent manner.

Invasion Assay (Matrigel Assay)

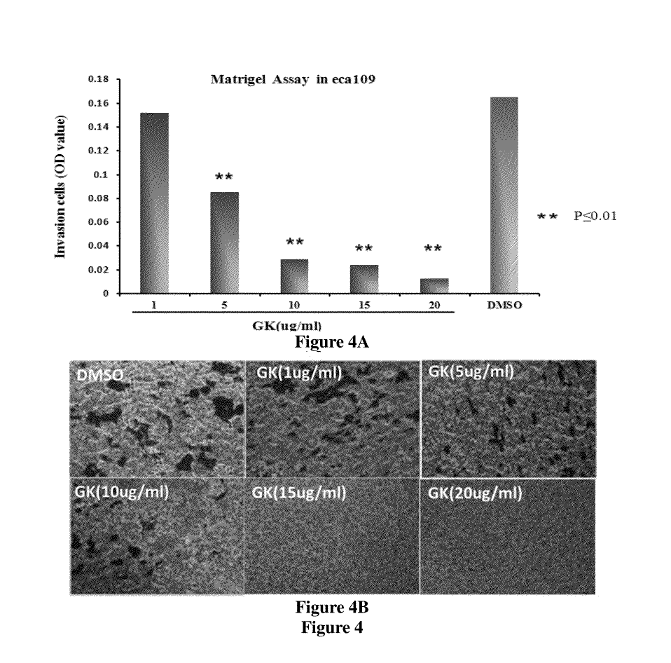

Cell invasion is determined using Matrigel (BD) coated transwell (Corning) with a pore size of 8 μm. 5×10⁴ cells are seeded in serum-free medium in the upper chamber, while medium containing 10% FBS in the lower chamber. After incubating for 72 hours at 37° C., cells in the upper chamber are carefully removed with a cotton swab and the cells that had traversed to reverse face of the membrane are fixed in methanol, stained with Giemsa, and counted. FIG. 4 shows the Guttiferone K suppresses ECA109 cells invasion through matrigel at a concentration dependent manner.

Plant Material

The pericarp of Garcinia yunnanensis Hu are collected in Luxi of Dehong prefecture, Yunnan province, China in 2006. The plant material is identified by Dr. Chunfeng Qiao. A herbarium sample is deposited in the Shanghai University of Traditional Chinese Medicine.

Effect of Guttiferone K on HCC Metastasis In Vitro and In Vivo

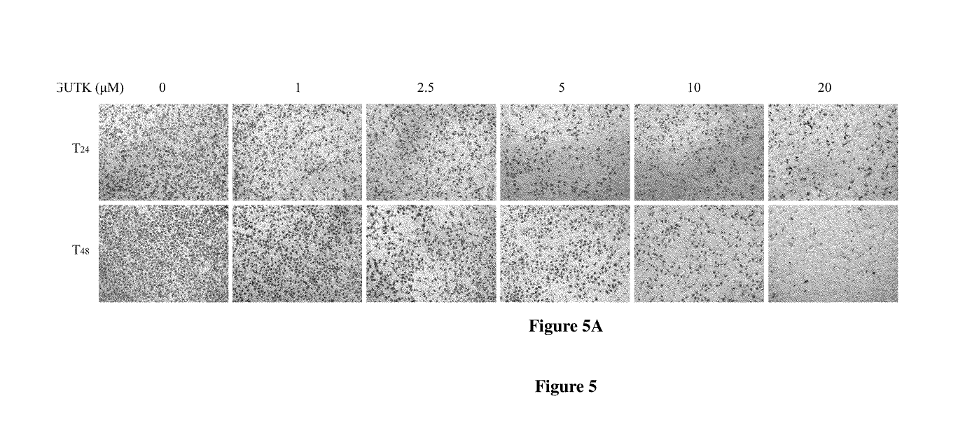

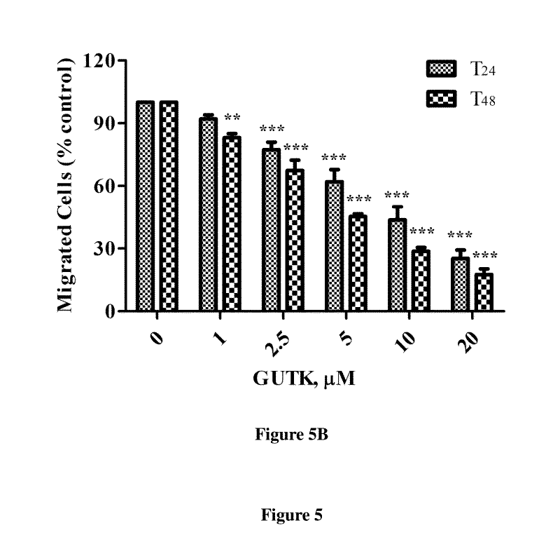

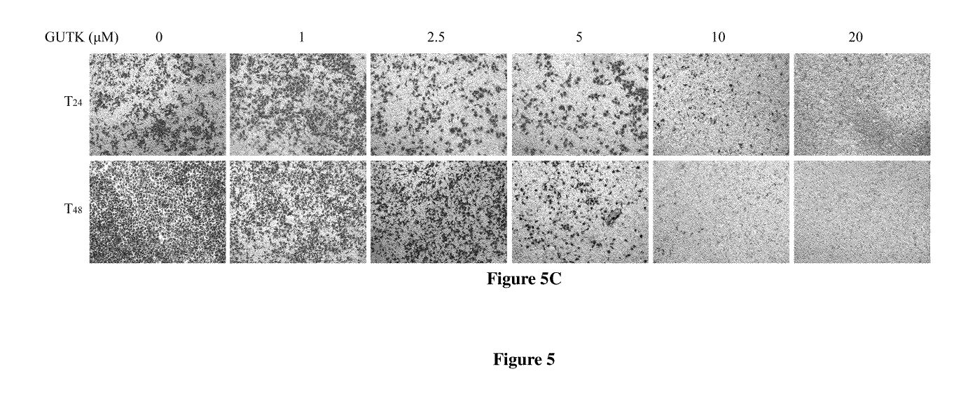

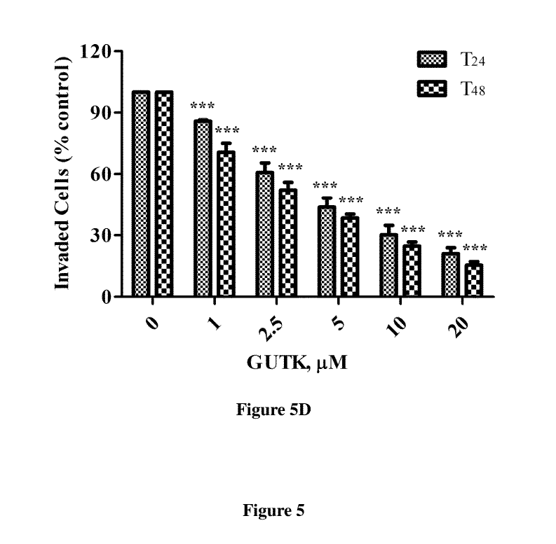

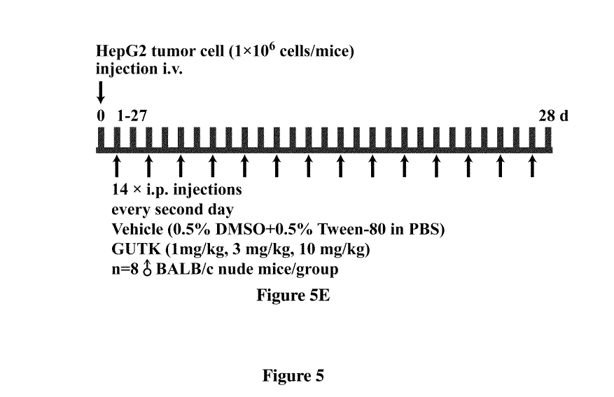

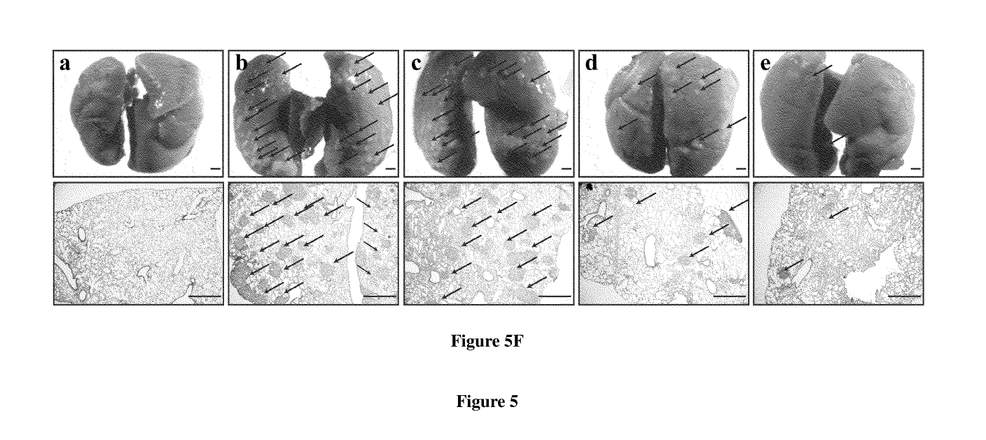

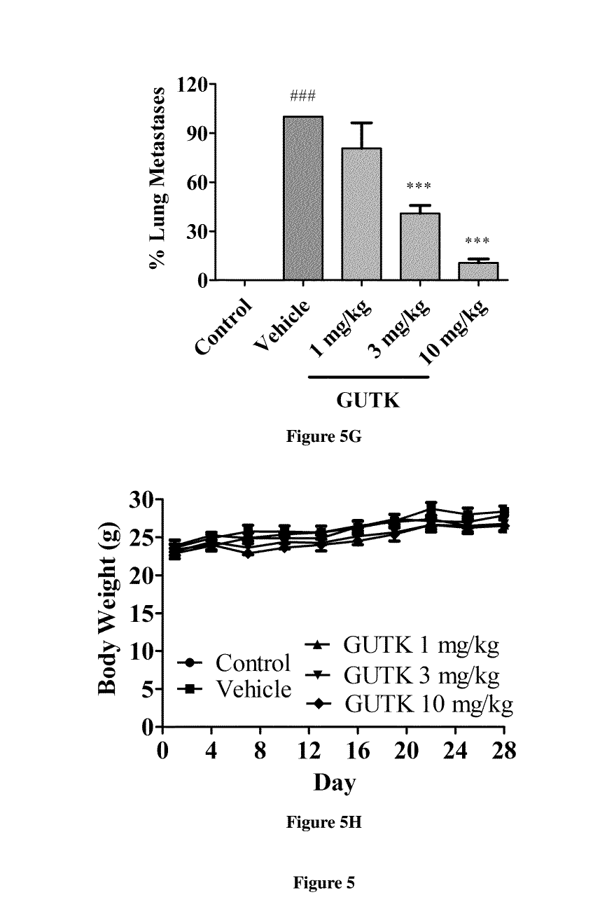

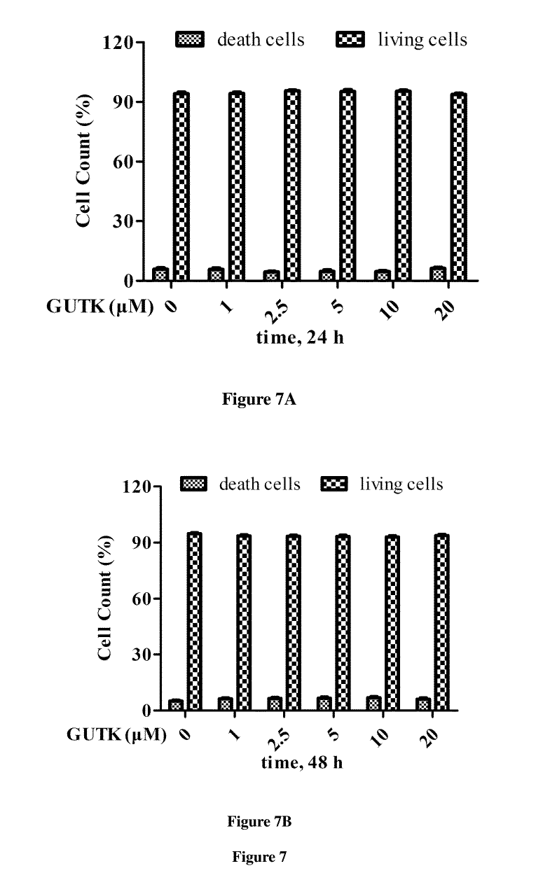

It has been reported in previous studie(s) that traditional Chinese herb Garcinia yunnanensis possesses therapeutic effects in cancer treatments. Therefore, several compounds from the plant are isolated and tested their effect on hepatic cancer cell migration (unpublished observations), an important feature of cancer metastasis. In migration assay, one of those tested compounds, known as Guttiferone K (the chemical structure shown in FIG. 1), reduced the motility of HepG2 cells (human liver carcinoma) at a minimal effective concentration of 1 μM, and such inhibition is in a concentration and time-dependent manner (FIG. 5A (microscope images) and FIG. 5B (bar chart)). Likewise, Guttiferone K treatment is also able to suppress invaded cell numbers in the matrigel-coated transwell invasion assay, and the half maximal inhibitory concentration of Guttiferone K is approximately 2.5 μM (FIG. 5C (microscope images) and FIG. 5D (bar chart)). Guttiferone K represents no cytotoxicity on HepG2 cells under the tested concentrations and during treatment time periods, which are measured by cell counting assay (FIG. 7A, FIG. 7B).

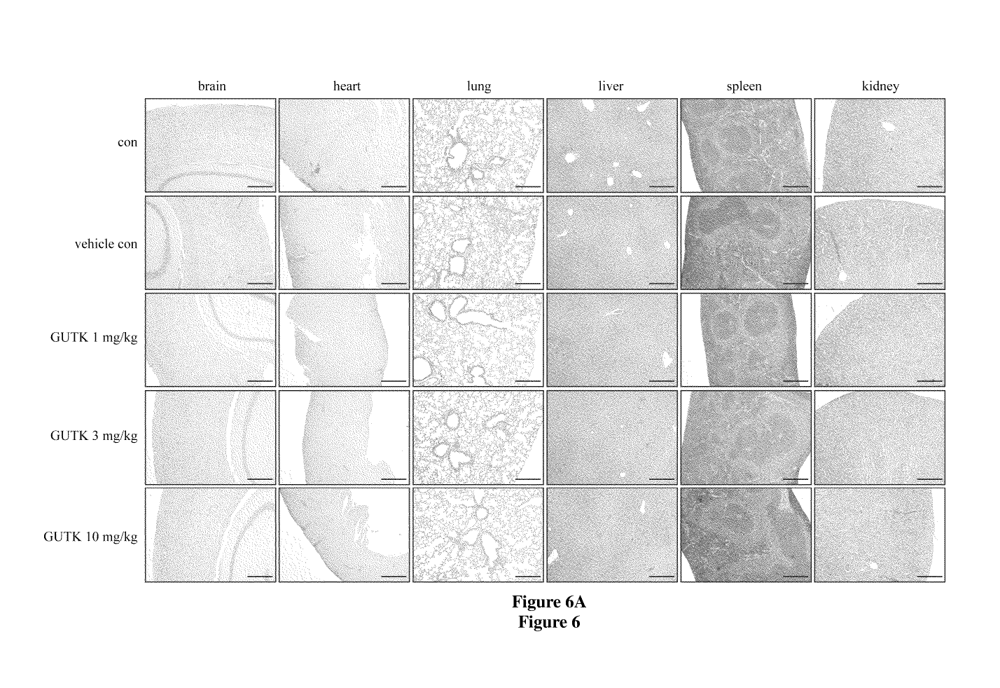

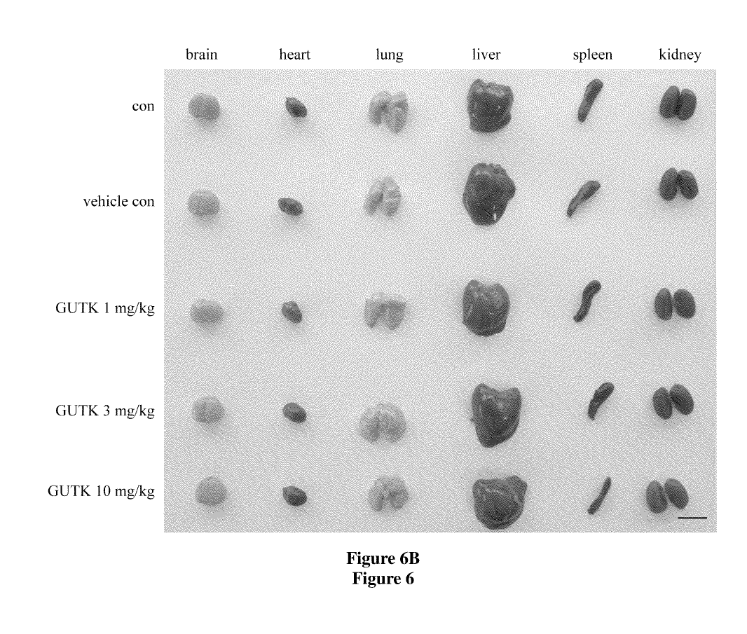

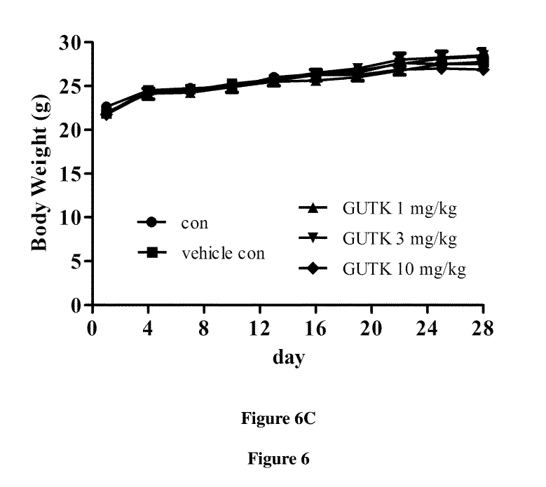

To study the potential effect of Guttiferone K on HCC metastasis, HepG2 cells injected via mouse tail vein are spontaneously metastasized to the lung region and formed tumor nodules in the model group, and Guttiferone K is immediately administrated to the mice after tumor cell challenge (the schedule shown in FIG. 5E). After 28 days, lungs are removed, fixed in Bouin's solution (FIG. 5F upper panel), and the tumor nodules are counted. The data shows that the number of metastasized nodules in the lungs of mice treated with Guttiferone K at 3 mg/kg and 10 mg/kg is reduced by 59.06% (95% CI=46.45% to 71.67%; p<0.01) and 89.43% (95% CI=83.35% to 95.51%; p<0.001), respectively, compared with vehicle group (FIG. 5G). However, administration of Guttiferone K at 1 mg/kg has little effect on HCC metastasis. There is no difference in body weight between the vehicle and the Guttiferone K-treated groups (FIG. 5H). Additionally, there is no evidence of organ size and cell morphology of target organs, including brain, heart, lung, liver, spleen, and kidney among the untreated (Control), the vehicle-treated (Vehicle), and the Guttiferone K-treated groups (FIG. 6A and FIG. 6B). Body weight shows no significant change during the 28-day treatment period (FIG. 6C). Altogether, these results indicate that Guttiferone K possesses an inhibitory effect on tumor cell migration and invasion, with little effect on tumor cell proliferation, which further confirm Guttiferone K suppresses tumor metastasis without any apparent cytotoxicity. The dosage of Guttiferone K for human is 70 mg/person (70 kg), which is equal to 10 mg/kg in our experiment, so 21 mg/person (70 kg) and 7 mg/person (70 kg) is equal to 3 mg/kg and 1 mg/kg, respectively, according to the dose translation from animal to human as specified in “Dose translation from animal to human studies revisited.” Shannon Reagan-Shaw, Minakshi Nihal, Nihal Ahmad FASEB J. 2008 March; 22(3): 659-661. Published online 2007 Oct. 17. doi: 10.1096/fj.07-9574LSF.

Discussion

In this invention, a compound, Guttiferone K, having a chemical structure of Formula I with anti-migration and anti-invasion effects on cancer cells is provided. Kan, W. L.; Yin, C.; Xu, H. X.; Xu, G.; To, K. K.; Cho, C. H.; Rudd, J. A.; Lin, G., Antitumor effects of novel compound, Guttiferone K, on colon cancer by p21Waf1/Cip1-mediated G(0)/G(1) cell cycle arrest and apoptosis. Int J Cancer 2012, shows that Guttiferone K has low toxicity in 10 mg/Kg concentration in mice. The present invention provides an anti-esophageal and anti-liver cancer composition comprising Guttiferone K (the chemical structure is represented by Formula I) and method of using the same for treating esophageal and liver cancer.

In one aspect of the present invention, there is provided a method for treating cancer comprising administering to a subject in need of a composition comprising an effective amount of a compound with the chemical structure of Formula I:

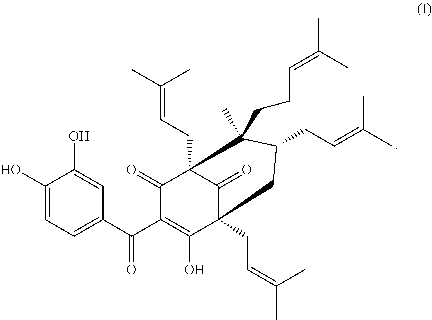 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]

In one embodiment, said compound is Guttiferone K.

In another embodiment, the cancer being treated comprises esophageal cancers and liver cancers.

In yet another embodiment, the compound has anti-cancer activities comprising anti-migration of cancer cells and anti-invasion of cancer cells.

In other embodiment, the effective amount of the compound of Formula I administered to said subject is more than 1 mg per 10 kg of the subject's body weight.

In a further embodiment, the effective amount of the compound of Formula I administered to said subject ranges from 3 mg per 10 kg to 10 mg per 10 kg of the subject's body weight.

In still another embodiment, the composition is administered via injection, including intraperitoneal, intravenous and subcutaneous injection.

In another aspect of the present invention there is provided a method of using the compound of Formula I for cancer treatment.

In one embodiment, said compound is used as a cancer metastasis inhibitor.

In another embodiment, the cancer being treated comprises esophageal cancers and liver cancers.

In an exemplary embodiment, the cancer treated is metastatic.

### INDUSTRIAL APPLICABILITY

The present invention discloses a chemical entity isolated from natural sources for its therapeutic uses. More particularly, it relates to compound that is naturally occurring in the plant of Garcinia yunnanensis Hu and its biological activity of anticancer effects.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A method for treating cancer comprising administering a subject in need of a composition comprising an effective amount of a compound with a chemical structure of Formula I, 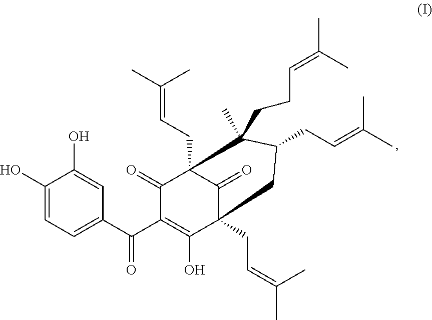 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]
wherein the composition comprises more than 1 mg of the compound of Formula I per 10 kg of the subject's body weight.

2. The method according to claim 1 wherein said compound is Guttiferone K.

3. The method according to claim 1 wherein the cancer comprises esophageal cancers and liver cancers.

4. The method according to claim 1 wherein the compound has anti-cancer activities comprising anti-migration and anti-invasion of cancer cells.

5. The method according to claim 1 wherein the effective amount of said compound ranges from 3 mg per 10 kg to 10 mg per 10 kg of the subject's body weight.

6. The method according to claim 1 wherein the composition is administered via intraperitoneal, intravenous or subcutaneous injection

7. A method of using a compound of Formula I, 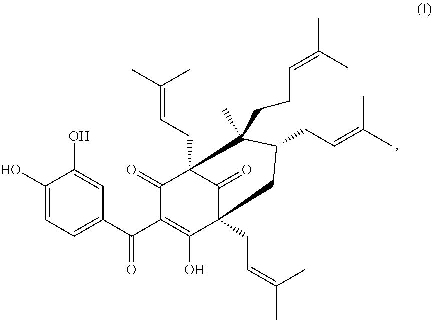 [SMILES: CC(C)=CCC[C@@]1(C)[C@H](CC=C(C)C)C[C@]2(CC=C(C)C)C(=O)[C@@]1(CC=C(C)C)C(=O)C(C(=O)c1ccc(O)c(O)c1)=C2O]
for metastatic cancer treatment.

8. The method according to claim 7 wherein said compound is a cancer metastasis inhibitor.

9. The method according to claim 7 wherein the cancer treated comprising esophageal cancers and liver cancers.

10. The method according to claim 7 wherein said compound is administered to a subject in need thereof in a range from 3 mg per 10 kg to 10 mg per 10 kg of the subject's body weight.

11. The method according to claim 7 wherein said compound is administered to a subject in need thereof at about 1 mg per 10 kg of the subject's body weight.

12. The method according to claim 7 wherein said compound is administered in a form of solution via intraperitoneal, intravenous, and/or subcutaneous injection.
